(19) United States
(12) Patent Application Publication
(10) Pub. No.: US 2026/0248695 A1
(43) Pub. Date: Aug. 27, 2026

(54) METHOD FOR PRODUCING A COSMETIC WATER-IN-OIL EMULSION FROM A GLYCERIN-IN-OIL EMULSION

(71) Applicant: BEIERSDORF AG, Hamburg (DE)

(72) Inventors: Kerstin SKUBSCH, Prisdorf (DE); Christal WALTER, Winsen/Luhe (DE); Julia ECKERT, Hamburg (DE)

(21) Appl. No.: 18/995,417

(22) PCT Filed: Jul. 11, 2023
(86) PCT No.: PCT/EP2023/069125

§ 371 Date: Jan. 16, 2025

(30) Foreign Application Priority Data
Jul. 21, 2022 (DE) 10 2022 207 466.1

(51) Int. Cl. A61K 8/06; A61K 8/23; A61K 8/34; A61K 8/37; A61K 8/85; A61K 8/92; A61Q 19/10

(52) U.S. Cl. CPC A61K 8/064; A61K 8/23; A61K 8/342; A61K 8/345; A61K 8/375; A61K 8/85; A61K 8/922; A61Q 19/10; A61K 2800/591

(57) ABSTRACT

A method for producing a cosmetic water-in-oil emulsion, wherein a glycerol-in-oil emulsion containing a) glycerol, b) one or more lipids liquid at room temperature and standard pressure, c) one or more waxes, d) one or more salts, e) polyglyceryl 3-polyricinoleate (INCI: Polyglyceryl-3-Polyricinoleate), is admixed with water in a storage container and shaken or stirred.

The present invention relates to a method for producing a cosmetic water-in-oil emulsion from a cosmetic glycerol-in-oil emulsion containing glycerol, one or more lipids liquid at room temperature and standard pressure, one or more waxes, salts and polyglyceryl 3-polyricinoleate (INCI: Polyglyceryl-3-Polyricinoleate).

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. It is common for these to comprise added active ingredients which are intended to regenerate the skin and for example to prevent and reduce the premature aging thereof (e.g. the appearance of fine lines and wrinkles) or protect against the adverse effects of UV radiation. Skincare products are typically offered in the form of emulsions where an outer water phase contains stable finely distributed oil droplets (O/W emulsion) or a continuous oil phase contains stable finely distributed water droplets (W/O emulsion).

Emulsions for skincare are generally marketed as ready-to-use products. However, these products have the disadvantage that they have a high weight due to their more or less high water content. Especially during transport the high weight leads to a relatively high energy and fuel consumption.

It is accordingly an object of the present invention to develop a cosmetic emulsion where the weight of the preparation is reduced and which, during transport especially of larger container quantities, results in lower energy and/or fuel consumption.

Most cosmetic emulsions are produced by the industrial manufacturer of the products using the “hot-hot” or “hot-cold” process, where an oil phase, which is generally at a temperature of more than 80° C., is blended with a likewise heated (“hot-hot”) or room-temperature (“hot-cold”) water phase with stirring, homogenized and subsequently cooled. All these manufacturing steps are associated with a relatively high energy consumption. It is accordingly an object of the present invention to develop a cosmetic emulsion where the energy consumption during industrial production is reduced.

The achievement of both objects should moreover markedly reduce the “CO₂ footprint”, i.e. the CO₂ emissions associated with the production and transport of the products.

Not least, conventional cosmetic finished products are often perceived by consumers as boring to use. Conventional finished products can meet the desire (or else the “play instinct”) of many consumers to themselves play a part in the production of cosmetics and the general consumer desire for products having at most a low “CO₂ footprint” to only a limited extent.

It is accordingly an object of the present invention to develop a cosmetic product which meets these consumer desires.

These objects are surprisingly achieved by a method for producing a cosmetic water-in-oil emulsion, wherein a glycerol-in-oil emulsion containing
a) glycerol,
b) one or more lipids liquid at room temperature and standard pressure,
c) one or more waxes,
d) one or more salts,
e) polyglyceryl 3-polyricinoleate (INCI: Polyglyceryl-3-Polyricinoleate),
is admixed with water in a storage container and shaken or stirred.

The glycerol-in-oil emulsion according to the invention allows the user/consumer to produce a stable, ready-to-use W/O emulsion for skin and body care by simple addition of water with a short period of shaking without having to use a technically complex or time-intensive production process.

Although there has been no lack of attempts in the past to develop low-water or even water-free “pre-emulsions” and offer them to the consumer for production of a care emulsion in the home environment, production was relatively complex either because it was necessary, for example, to use warm water or because the use of complex equipment such as agitators (“mixers”) was required. Or else the end product produced at home was left wanting in terms of quality because a really finely dispersed homogeneous emulsion was not formed. The regionally varying salt content of the water (for example the “water hardness”) also posed problems since this has a significant influence on the formation and stability of an emulsion. What worked in Hamburg could not necessarily be expected to succeed in Munich.

In the context of the present disclosure the terms “according to the invention” etc. always refer to the process according to the invention and to the product of the process.

It is particularly advantageous according to the invention for the process according to the invention when the preparation contains no further water in addition to the water present in the glycerol and the salts. An additional small content of water can destabilize the glycerol-in-oil emulsion and lead to phase separation. A stable W/O emulsion system is re-formed only at higher water concentrations (greater than 30% by weight) as occurring in W/O emulsion produced by the process according to the invention.

It is therefore preferable according to the invention when the employed glycerol contains not more than 1% by weight, based on the glycerol, of water. Ideally 99% or 99.5% pure glycerol is employed.

It is advantageous according to the invention when the salts employed in the glycerol-in-oil emulsion are alkaline earth metal salts.

Embodiments of the present invention preferred according to the invention are characterized in that the salt employed in the glycerol-in-oil emulsion is magnesium sulfate.

It is moreover particularly preferable according to the invention when the magnesium sulfate employed is magnesium sulfate heptahydrate (MgSO₄·7H₂O). This may appear surprising at first glance but anhydrous magnesium sulfate is markedly more difficult to incorporate into the glycerol-in-oil emulsion than magnesium sulfate containing water of crystallization. Anhydrous magnesium sulfate is less easily dissolvable in glycerol and tends to form lumps.

It is advantageous according to the invention when the employed lipids liquid at room temperature and standard pressure are one or more compounds selected from the group caprylic/capric acid triglyceride (INCI: Caprylic/Capric Triglyceride), ethylhexyl stearate (INCI: Ethylhexyl Stearate), triisostearin (INCI: Triisostearin), coco caprylate/caprate (INCI: Coco-Caprylate Caprate), octyldodecanol, coco glycerides (INCI: Coco Glycerides), vegetable oil (INCI: Vegetable Oil), dicaprylyl ether, sunflower oil (INCI: Helianthus Annuus Seed Oil).

The use of caprylic/capric acid triglyceride (INCI: Caprylic/Capric Triglyceride), ethylhexyl stearate (INCI: Ethylhexyl Stearate) and triisostearin (INCI: Triisostearin) is preferable according to the invention.

When the oil phase of the glycerol-in-oil emulsion contains caprylic/capric acid triglyceride (INCI: Caprylic/Capric Triglyceride) it is preferable according to the invention when this compound is employed in a concentration of 10% to 50% by weight based on the total weight of the glycerol-in-oil emulsion.

When the oil phase of the glycerol-in-oil emulsion contains ethylhexyl stearate (INCI: Ethylhexyl Stearate) it is preferable according to the invention when this compound is employed in a concentration of 10% to 50% by weight based on the total weight of the glycerol-in-oil emulsion.

When the oil phase of the glycerol-in-oil emulsion contains triisostearin (INCI: Triisostearin) it is preferable according to the invention when this compound is employed in a concentration of 10% to 50% by weight based on the total weight of the glycerol-in-oil emulsion.

It is advantageous according to the invention when the wax employed is one or more compounds selected from the group of hydrogenated rapeseed oil (INCI: Hydrogenated Rapeseed Oil), hydrogenated castor oil (INCI: Hydrogenated Castor Oil), hydrogenated vegetable oil (INCI: Hydrogenated Vegetable Oil), glyceryl stearate (INCI: Glyceryl Stearate). The use of hydrogenated rapeseed oil (INCI: Hydrogenated Rapeseed Oil) is preferable according to the invention.

When the oil phase of the glycerol-in-oil emulsion contains hydrogenated rapeseed oil (INCI: Hydrogenated Rapeseed Oil) it is preferable according to the invention when this compound is employed in a concentration of 0.5% to 5% by weight based on the total weight of the glycerol-in-oil emulsion.

The embodiments of the present invention that are advantageous according to the invention are characterized in that the preparation comprises the glycerol in an amount of 20% to 30% by weight based on the total weight of the glycerol-in-oil emulsion.

The embodiments of the present invention that are advantageous according to the invention are further characterized in that the preparation contains the lipids liquid at room temperature and standard pressure of feature b) in a total amount of 40% to 60% by weight based on the total weight of the glycerol-in-oil emulsion.

It is further advantageous according to the invention when the preparation contains waxes of feature c) in an amount of 0.5% to 5% by weight based on the total weight of the glycerol-in-oil emulsion.

It is further advantageous according to the invention when the preparation contains magnesium sulfate in an amount of 1% to 4% by weight based on the total weight of the glycerol-in-oil emulsion.

Embodiments of the present invention that are advantageous according to the invention are not least characterized in that the preparation comprises polyglyceryl 3-polyricinoleate (INCI: Polyglyceryl-3-Polyricinoleate) in an amount of 10% to 20% by weight based on the total weight of the glycerol-in-oil emulsion.

The glycerol-in-oil emulsion according to the invention may further contain additional ingredients, such as are typically employed in cosmetic emulsions, for example cosmetic active ingredients, UV filters, preservatives, antioxidants, powder raw materials etc.

It is advantageous according to the invention when the glycerol-in-oil emulsion according to the invention is contained in a jar or a bottle having a water-impermeable closure made of tinplate, glass, polypropylene, polyethylene or polyethylene terephthalate. The fill level in this storage container should preferably according to the invention be selected such that this storage container can additionally accommodate three times the amount of water.

It is advantageous according to the invention for the process according to the invention when the employed storage container is ajar or a bottle made of tinplate, glass, polypropylene, polyethylene or polyethylene terephthalate having a water-impermeable closure and an emulsion is formed by shaking.

Embodiments advantageous according to the invention of the process according to the invention are further characterized in that the process is performed at a temperature between 10° C. and 30° C.

It is further advantageous for the process according to the invention when the weight ratio of glycerol-in-oil emulsion to water is from 25:75% by weight to 60:40% by weight. It is preferable according to the invention when the weight ratio of glycerol-in-oil emulsion to water is from 30:70% by weight to 50:50% by weight and particularly preferably 2:1.

The usage amount of the water makes it possible to control the viscosity of the W/O emulsion obtainable from the glycerol-in-oil emulsion by addition of water. A low content of water (60-75% by weight) results in a high-viscosity preparation, in particular in a cream. By contrast, smaller amounts of water (40-60% by weight) form lower-viscosity lotions.

In addition, it is advantageous according to the invention when the water has a temperature of at least 10° C. A water temperature of 15° C. to 25° C. is preferable according to the invention.

It is not least a feature of the embodiments advantageous according to the invention of the process according to the invention that the shaking of the storage container is carried out by hand and the shaking process has a duration of not more than 90 seconds. It is preferable according to the invention when the shaking process has a duration of 10 to 30 seconds.

The present invention also provides a cosmetic water-in-oil emulsion produced by the process according to the invention.

Advantageously according to the invention said emulsion is characterized in that it is in the form of a cream or lotion.

### Comparative Experiments

The effect according to the invention was demonstrated by way of example with the following experiment:

The following concentrates (=glycerol-in-oil emulsion) and the activation time until formation of the water-in-oil emulsion (=method product) were investigated.

### Comparative Experiments—Emulsifiers

| | Concen- | Concen- | Concen- | Concen- | Concen- |
|---|---|---|---|---|---|
| INCI (amount in % | trate | trate | trate | trate | trate |
| by wt.) | G11 | G12 | G13 | G14 | G78 |
| Polyglyceryl-3- | 14 | | | | 14 |
| Polyricinoleate | | | | | |
| Diisostearoyl | | 14 | | | |
| Polyglyceryl-3 Dimer | | | | | |
| Dilinoleate | | | | | |
| Polyglyceryl-4 | | | 14 | | |
| Diisostearate/ | | | | | |
| Polyhydroxystearate/ | | | | | |
| Sebacate | | | | | |
| Polyglyceryl-3 | | | | 14 | |
| Diisostearate + Aqua | | | | | |
| Polyglyceryl-2 | | | | | 14 |
| Dipolyhydroxystearate | | | | | |
| Caprylic/Capric | 19 | 19 | 19 | 19 | 19 |
| Triglyceride | | | | | |
| Ethylhexyl Stearate | 17 | 17 | 17 | 17 | 17 |
| Hydrogenated Rapeseed | 3 | 3 | 3 | 3 | 3 |
| Oil | | | | | |
| Triisostearin | 17 | 17 | 17 | 17 | 17 |
| Parfum | | | | | 1 |
| Glycerol | 28 | 28 | 28 | 28 | 28 |
| Magnesium Sulfate | 3 | 3 | 3 | 3 | 3 |
| Concentrate viscosity | 550 | 2600 | 550 | 550 | 700 |
| after 1 day | mPas | mPas | mPas | mPas | mPas |
| Concentrate stability | OK | OK | Signifi- | Signifi- | Signifi- |
| after 1 day at 25° C. | | | cant oil | cant oil | cant oil |
| | | | separa- | separa- | separa- |
| | | | tion | tion | tion |
| Concentrate stability | OK | Some | Signifi- | Signifi- | Signifi- |
| after 7 days at 25° C. | | oil | cant oil | cant oil | cant oil |
| | | separa- | separa- | separa- | separa- |
| | | tion | tion | tion | tion |
| Activation time for W/O | 17 | 19 | 140 | 80 | 9 |
| emulsion formation | seconds | seconds | seconds | seconds | seconds |
| with 30% concentrate | | | | | |
| and 70% tap water | | | | | |

### Production of Concentrate (=Glycerol-In-Oil Emulsion) in the Laboratory:

The emulsifiers and lipid-soluble raw materials in the oil phase are melted at 80° C. prior to homogenization until all constituents have attained the liquid state. Constituents of the hydrophilic phase are dissolved in the glycerol using a heatable magnetic stirrer at 75° C. with stirring until the liquid is clear. This glycerol phase is added to the oil phase during the homogenization process (for example with an IKA T25 digital Ultra-Turrax disperser) as soon as both phases have attained a temperature of 75-80° C. The phases are combined at a speed of 8000 rpm. Once the phases have been combined, homogenization is performed at a speed of 11 200 rpm for a further 3 min. The concentrate is then cooled with stirring using the IKA stirrer and a second homogenization is optionally carried out after addition of a fragrance at about 35° C.

### Production of the W/O Emulsion and Determination of the Activation Time:

In order to produce the water-containing W/O emulsions and to determine the activation time until formation thereof initially the concentrate (glycerol-in-oil emulsion) and subsequently the water are weighed into a 500 ml wide-necked flask in a total amount of 400 g (for example at 30% to 70%: 120 g of concentrate and 280 g of water). The vessel is then securely closed and shaken by hand until the resulting W/O emulsion has a homogeneous appearance and the time is stopped.

If no homogeneous W/O emulsion is formed within 3 minutes of shaking, the batch is terminated.

### Comparative Experiments—Waxes

| INCI (amount | Concentrate | Concentrate | Concentrate | Concentrate | Concentrate | Concentrate |
|---|---|---|---|---|---|---|
| in % by wt.) | G21 | G22 | G23 | G24 | G25 | G26 |
| Polyglyceryl-3- | 13.9 | 13.9 | 13.9 | 13.9 | 13.9 | 13.9 |
| Polyricinoleate | | | | | | |
| Caprylic/Capric | 19.4 | 19.4 | 19.4 | 19.4 | 19.4 | 19.4 |
| Triglyceride | | | | | | |
| Ethylhexyl | 16.7 | 16.7 | 16.7 | 16.7 | 16.7 | 16.7 |
| Stearate | | | | | | |
| Triisostearin | 16.7 | 16.7 | 16.7 | 16.7 | 16.7 | 16.7 |
| Hydrogenated | 2.8 | | | | | |
| vegetable oil | | | | | | |
| Cetyl Alcohol | | 2.8 | | | | |
| Glyceryl | | | 2.8 | | | |
| Stearate | | | | | | |
| Cetyl Palmitate | | | | 2.8 | | |
| Hydrogenated | | | | | 2.8 | |
| Coco- | | | | | | |
| Glycerides | | | | | | |
| Myristyl | | | | | | 2.8 |
| Myristate | | | | | | |
| Glycerol | 27.8 | 27.8 | 27.8 | 27.8 | 27.8 | 27.8 |
| Magnesium | 2.8 | 2.8 | 2.8 | 2.8 | 2.8 | 2.8 |
| Sulfate | | | | | | |
| Activation time | 17 seconds | >3 minutes | 120 seconds | >3 minutes | >3 minutes | >3 minutes |
| for W/O | | | | | | |
| emulsion | | | | | | |
| formation | | | | | | |
| with 30% | | | | | | |
| concentrate | | | | | | |
| and 70% tap | | | | | | |
| water | | | | | | |

| INCI (amount | Concentrate | Concentrate | Concentrate | Concentrate | Concentrate |
|---|---|---|---|---|---|
| in % by wt.) | G27 | G28 | G30 | S147 | S158 |
| Polyglyceryl-3- | 14.1 | 13.9 | 13.9 | 13.9 | 13.9 |
| Polyricinoleate | | | | | |
| Caprylic/Capric | 19.7 | 19.4 | 19.4 | 19.4 | 20.5 |
| Triglyceride | | | | | |
| Ethylhexyl | 16.9 | 16.7 | 16.7 | 16.7 | 17.5 |
| Stearate | | | | | |
| Triisostearin | 16.9 | 16.7 | 16.7 | 16.7 | 17.5 |
| Hydrogenated | 1.4 | | 1.4 | | |
| vegetable oil | | | | | |
| Butyrospermum | | 2.8 | | | |
| Parkii Butter | | | | | |
| Hydrogenated | | | 1.4 | 2.8 | |
| Rapeseed Oil | | | | | |
| Glycerol | 28.2 | 27.8 | 27.8 | 27.8 | 27.8 |
| Magnesium | 2.8 | 2.8 | 2.8 | 2.8 | 2.8 |
| Sulfate | | | | | |
| Activation time | 90 seconds | 180 seconds | 35 seconds | 10 seconds | 150 seconds |
| for W/O | | | | | |
| emulsion | | | | | |
| formation | | | | | |
| with 30% | | | | | |
| concentrate and | | | | | |
| 70% tap water | | | | | |

### Comparative Experiments—Magnesium Sulfate/Wax

Composition of the W/G emulsion produced from a concentrate by shaking with the appropriate amount of water.

| | W/O | | W/O | | W/O | |
|---|---|---|---|---|---|---|
| | emulsion | | emulsion | | emulsion | |
| INCI (amount in % by wt.) | SB 19 | | SB 20 | | SB 22 | |
| Polyglyceryl-3- | 5 | | 5 | | 5 | |
| Polyricinoleate | | | | | | |
| Caprylic/Capric | 7 | | 7 | | 7 | |
| Triglyceride | | | | | | |
| Ethylhexyl Stearate | 6 | | 6 | | 6 | |
| Hydrogenated Rapeseed Oil | 1 | | 1 | | | |
| Triisostearin | 6 | | 6 | | 6 | |
| Glycerol | 10 | | 10 | | 10 | |
| Magnesium Sulfate | 1 | | | | 1 | |
| Aqua | 64 | | 65 | | 65 | |
| Droplet size Mastersizer | 3.5 | μm | 9.4 | μm | 6.7 | μm |
| d 43 | | | | | | |
| Activation time | 12 | seconds | 12 | seconds | 150 | seconds |

### EXAMPLES

### Production of the Concentrate (Glycerol-In-Oil Emulsion)

The usage amounts are derivable from the exemplary formulations in the table.

The fat phase comprising Polyglyceryl-3-Polyricinoleate, Caprylic/Capric Triglyceride, Ethylhexyl Stearate, Hydrogenated Rapeseed Oil and Triisostearin is weighed into a beaker and heated to 80° in a water bath with repeated stirring using a stirring thermometer. The magnesium sulfate is weighed into a beaker together with the glycerol and dissolved until clear using a magnetic stirrer on a hot plate at 75° C. with vigorous stirring. The hot glycerol phase is dispersed into the hot fat phase using an Ultra-Turrax disperser at 8000 rpm for 2 minutes. The mixture is then dispersed at 11 200 rpm for a further 3 minutes. This is followed by cooling to 35° C. using an IKA stirrer. The fragrance is stirred into the cooled glycerol-in-oil emulsion and the mixture is then homogenized again for 2 minutes with the Ultra-Turrax disperser at 11 200 rpm.

| | Concentrate | |
|---|---|---|
| INCI in g | G55 | |
| Polyglyceryl-3-Polyricinoleate | 90 | g |
| Caprylic/Capric Triglyceride | 126 | g |
| Ethylhexyl Stearate | 108 | g |
| Hydrogenated Rapeseed Oil | 18 | g |
| Triisostearin | 108 | g |
| Parfum | 6.3 | g |
| Glycerol | 180 | g |
| Magnesium Sulfate | 18 | g |
| | 654.3 | g |

The same concentrate may be used to produce emulsions of differing viscosity depending on the amount of water selected. Examples for the resulting W/O emulsions follow.

Production of the W/O emulsion and determination of the activation time in the laboratory: In order to produce the water-containing W/O emulsions and to determine the activation time initially the concentrate (glycerol-in-oil emulsion) and subsequently the tap water are weighed into a 500 ml wide-necked flask in a total amount of 400 g (for specific amounts see table). The vessel is then firmly secured and shaken by hand (as for example in cocktail mixing) until the emulsion has a homogeneous appearance—the time is stopped until W/O emulsion formation is complete.

If no homogeneous emulsion is formed within 3 minutes of shaking, the batch is terminated.

| | W/O | | W/O | | W/O | |
|---|---|---|---|---|---|---|
| | emulsion | | emulsion | | emulsion | |
| | T13 | | T14 | | T15 | |
| Glycerol-in-oil | 120 | g | 160 | g | 200 | g |
| emulsion G55 | | | | | | |
| Tap water 20° C. | 280 | g | 240 | g | 200 | g |
| Activation time for | 8 | seconds | 5 | seconds | 3 | seconds |
| W/O emulsion | | | | | | |
| formation | | | | | | |

| | | | |
|---|---|---|---|
| Viscosity at 25° C. | 14 000 | 10 000 | 4600 |
| Rheomat 123 | mPas | mPas | mPas |

The following table lists the ingredients of the aforementioned WIG emulsions:

| | W/O | W/O | W/O |
|---|---|---|---|
| | emulsion | emulsion | emulsion |
| INCI in % by wt. | T13 | T14 | T15 |
| Polyglyceryl-3-Polyricinoleate | 4.1 | 5.5 | 6.9 |
| Caprylic/Capric Triglyceride | 5.8 | 7.7 | 9.6 |
| Ethylhexyl Stearate | 5.0 | 6.6 | 8.3 |
| Hydrogenated Rapeseed Oil | 0.8 | 1.1 | 1.4 |
| Triisostearin | 5.0 | 6.6 | 8.3 |
| Parfum | 0.3 | 0.4 | 0.5 |
| Glycerol | 8.3 | 11.0 | 13.8 |
| Magnesium Sulfate | 0.8 | 1.1 | 1.4 |
| Aqua | 70 | 60 | 50 |
| Activation time for W/O emulsion | 8 seconds | 5 seconds | 3 seconds |
| formation | | | |
| Viscosity at 25° C. Rheomat 123 | 14 000 | 10 000 | 4600 |
| | mPas | mPas | mPas |

### Further Examples of Inventive Glycerol-In-Oil Emulsions:

| | Concen- | Concen- | Concen- | Concen- | Concen- |
|---|---|---|---|---|---|
| | trate | trate | trate | trate | trate |
| INCI in % by wt. | B 1 | B 2 | B 3 | B 4 | B 5 |
| Polyglyceryl-3- | 14.0 | 13.9 | 13.9 | 13.9 | 10.0 |
| Polyricinoleate | | | | | |
| Caprylic/Capric | 17.1 | 19.4 | 19.4 | 19.4 | 20.0 |
| Triglyceride | | | | | |
| Dicaprylyl Ether | 17.1 | | | | |
| Hydrogenated Rapeseed | 5.7 | 2.8 | 2.8 | 2.8 | 5.0 |
| Oil | | | | | |
| Hydrogenated vegetable | | | | | 5 |
| oil | | | | | |
| Triisostearin | 17.0 | 16.7 | 16.7 | 16.7 | |
| Octyldodecanol | | 16.7 | | | 10 |
| Helianthus Annuus | | | | | 20 |
| Hybrid Oil | | | | | |
| Coco-Caprylate/Caprate | | | 16.7 | | |
| Coco glycerides | | | | 16.7 | |
| Glycerol | 28.0 | 27.8 | 27.8 | 27.8 | 30.0 |
| Magnesium Sulfate | 1.0 | 2.8 | 2.8 | 2.8 | 2.5 |
| | Concen- | Concen- | Concen- | Concen- | Concen- |
| | trate | trate | trate | trate | trate |
| INCI in % by wt. | G 80 | G 87 | G 90 | G 92 | S 100 |
| Polyglyceryl-3- | 13.8 | 13.8 | 13.8 | 13.8 | 13.9 |
| Polyricinoleate | | | | | |
| Caprylic/Capric | 19.3 | | | | 16.7 |
| Triglyceride | | | | | |
| Ethylhexyl Stearate | 16.5 | | | | |
| Hydrogenated Rapeseed | 2.8 | 2.8 | 2.8 | 2.8 | 2.8 |
| Oil | | | | | |
| Triisostearin | 16.5 | | | | 19.4 |
| Coco glycerides | | 52.2 | | | |
| Vegetable Oil | | | 52.2 | | |
| Helianthus Annuus | | | | 52.2 | |
| Hybrid Oil + Lecithin + | | | | | |
| Ascorbyl Palmitate | | | | | |
| Dicaprylyl Ether + | | | | | 16.7 |
| Tocopherol | | | | | |
| Glyceryl Stearate | | | | | 2.8 |
| Parfum | 1.0 | 1.0 | 1.0 | 1.0 | |
| Glycerol | 27.5 | 27.5 | 27.5 | 27.5 | 27.8 |
| Calcium chloride | 2.8 | | | | |
| Magnesium Sulfate | | 2.8 | 2.8 | 2.8 | |

1.-21. (canceled)

22. A method for producing a cosmetic water-in-oil emulsion, wherein the method comprises admixing in a storage container water to a glycerol-in-oil emulsion comprising
(a) glycerol,
(b) one or more lipids liquid at room temperature and standard pressure,
(c) one or more waxes,
(d) one or more salts,
(e) polyglyceryl 3-polyricinoleate (INCI: Polyglyceryl-3-Polyricinoleate),
and shaking and/or stirring a mixture thus obtained.

23. The method of claim 22, wherein the glycerol-in-oil emulsion contains no further water in addition to water present in the glycerol and the salt.

24. The method of claim 22, wherein (d) comprises one or more alkaline earth metal salts.

25. The method of claim 22, wherein (d) comprises magnesium sulfate.

26. The method of claim 22, wherein (d) comprises magnesium sulfate heptahydrate (MgSO₄·7H₂O).

27. The method of claim 22, wherein the glycerol employed contains not more than 1% by weight of water, based on a weight of the glycerol.

28. The method of claim 22, wherein (b) comprises one or more of caprylic/capric acid triglyceride (INCI: Caprylic/Capric Triglyceride), ethylhexyl stearate (INCI: Ethylhexyl Stearate), triisostearin (INCI: Triisostearin), coco caprylate/caprate (INCI: Coco-Caprylate Caprate), octyldodecanol, coco glycerides (INCI: Coco Glycerides), vegetable oil (INCI: Vegetable Oil), dicaprylyl ether, sunflower oil ([NCI: Helianthus Annuus Seed Oil).

29. The method of claim 22, wherein (c) comprises one or more of hydrogenated rapeseed oil ([NCI: Hydrogenated Rapeseed Oil), hydrogenated castor oil (INCI: Hydrogenated Castor Oil), hydrogenated vegetable oil (INCI: Hydrogenated Vegetable Oil), glyceryl stearate (INCI: Glyceryl Stearate).

30. The method of claim 22, wherein the emulsion comprises from 20% to 30% by weight of glycerol, based on a total weight of the emulsion.

31. The method of claim 22, wherein the emulsion comprises from 40% to 60% by weight of (b), based on a total weight of the emulsion.

32. The method of claim 22, wherein the emulsion comprises from 0.5% to 5% by weight of (c), based on a total weight of the emulsion.

33. The method of claim 22, wherein the emulsion comprises from 1% to 4% by weight of magnesium sulfate, based on a total weight of the emulsion.

34. The method of claim 22, wherein the emulsion comprises from 10% to 20% by weight of (e), based on a total weight of the emulsion.

35. The method of claim 22, wherein the storage container is a jar or a bottle made of tinplate, glass, polypropylene, polyethylene or polyethylene terephthalate having a water-impermeable closure, and the emulsion is formed by shaking.

36. The method of claim 22, wherein the method is carried out at a temperature of from 10° C. to 30° C.

37. The method of claim 22, wherein a weight ratio of glycerol-in-oil emulsion to water is from 25:75 to 60:40.

38. The method of claim 22, wherein the water has a temperature of at least 10° C.

39. The method of claim 22, wherein the water has a temperature of from 15° C. to 25° C.

40. The method of claim 22, wherein shaking of the storage container is carried out by hand and lasts not more than 90 seconds.

41. A cosmetic water-in-oil emulsion, wherein the emulsion is produced by the method of claim 22.
